(19) United States
(12) Patent Application Publication
(10) Pub. No.: US 2020/0179667 A1
(43) Pub. Date: Jun. 11, 2020

(54) Apparatus for promoting hair growth

(71) Applicants: Ying Wang, Irvine, CA (US); Wei Wu, Shanghai (CN)

(72) Inventors: YING WANG, IRVINE, CA (US); Wei WU, Shanghai (CN)

(21) Appl. No.: 16/211,371

(22) Filed: Dec. 6, 2018

(51) Int. Cl. A61M 37/00; A61N 5/06; A61H 23/02

(52) U.S. Cl. CPC A61M 37/0015; A61N 2005/067; A61H 23/02; A61N 5/062

(57) ABSTRACT

A cap promoting hair growth is provided. In one embodiment, the cap covers the user's scalp while the hair growth is promoted by the vibration of micro-needle massage pad along with the illumination of laser beam with the growth factor applied to the user's scalp.

### BACKGROUND OF THE INVENTION

### 1. Field of the Invention

This invention relates to an apparatus for promoting hair growth, and more particularly a headwear with at least one microneedle pad for massage and stimulation of scalp where laser illumination and growth factor can be further included to give combined effects.

### 2. Description of the Related Art

Hair is formed by matrix cells within the hair follicle during a cycle of alternating periods of rest (telogen phase) and activity (anagen phase). It is during the relatively long anagen phase that follicular cells divide and produce hair. Once hair is produced the cycle progresses to the relatively short telogen phase during which cell division stops, hair growth ceases and the attachment of the hair to the follicle become weaker until the old hair is shed. The cycle repeats itself unless there is an interruption resulting from specific genetic factors, aging or disease. Interruption of the hair cycle may result in baldness or alopecia.

Balding is generally characterized by an increase in cycles with the anagen phase shortened with each cycle resulting in a rapid diminution in amount of hair and length of hair. The most common type of hair loss in humans, including both males and females, is termed androgenetic alopecia or male pattern baldness. Alopecia results from the gradual miniaturization of the long, coarse and pigmented terminal hairs to the very fine, non-pigmented and short vellus hairs due to the diminution in the anagen phase with repeated cycles. This transition in hair type is associated with and results from an alteration of the hair cycle. Baldness results in a diminution of the size of hair follicles without a decrease in follicular numbers. Small telogen follicles found in balding skin are not devoid of hair but contain the very fine vellus hairs. Consequently, baldness is not considered a degenerative phenomenon, but a series of progressive changes in the scalp which results in the conversion of terminal hairs to velus hairs.

Currently there is no proven pharmaceutical treatment for this diminution in the size of hair follicles which results in baldness, although a few agents has shown promise. In general, microneedle stimulation systems are well known for their use in skin care treatment of various conditions such as wrinkles, acne scarring, stretch marks, skin whitening and facial rejuvenation. In microneedling, a method of piercing holes in the skin and applying drugs or cosmetics to the skin provides a way to rapidly and sufficiently permeate the skin. Another advantage of using microneedles is to injure the skin just enough to begin natural healing processes and stimulate collagen and elastin production, and the like, to heal the skin. In these methods, hundreds to thousands of tiny holes or microconduits are created in the skin with the microneedling device without damaging the deeper layers of the skin. This injury to the skin begins a natural healing process that leads to the release of natural stimulants and growth factors which stimulates the formation of new natural collagen and elastin in the papillary dermis to produce new, healthy skin tissue. Also, new capillaries are formed. This neovascularisation and neocollagenesis associated with the wound healing process leads to the formation of younger looking skin, reduction of skin pathologies and improvement of scars. Generally called percutaneous collagen induction therapy, microneedling has also been used in the treatment of photo ageing. Furthermore, medical substances may be applied to the site where the holes are created and the substances are supposed to permeate into the skin through the tiny holes. Microneedling is generally applied to the face, neck, scalp, and just about anywhere on the body where a particular condition warrants without removing or permanently damaging the skin. Microneedling is simple and safe. A predetermined number of needles are inserted into the skin to the desired depth. As a reaction to the minor injury, the skin tissue begins a natural wound-healing cascade. This natural process forms new healthy dermal tissue that helps smooth scars, remove wrinkles and improve pigmentation, and yields a younger, healthier and a cleaner-looking skin.

U.S. Pat. No. 9,227,089 relates to a method and apparatus for promoting hair growth, in which hand-held micro needling roller to traumatizing the skin. U.S. Pat. No. 9,358,376 relates to a hand-held micro-needle roller infusion system for transdermal or intradermal delivery of active agents, skin-care products, and other topical formulations. Hand-held rollers with microneedles are also disclosed in U.S. Pat. Application 2011/0040236A1, 2015/0231382A1, 2010/0262081A1, 2011/0009782, 2009/0312691A1, and CN. Pat. 203379477U. There are other type of hand-held microneedling devices such as disclosed in U.S. Pat. Application 2010/0030152A1 and 2014/0079686.

One effect of microneedles is to penetrate completely through the stratum corneum. When the stratum corneum is penetrated, liquid drug or other active ingredient such as growth factors can be more efficient applied through the relatively tiny openings created by the microneedles. A major advantage provided by micro needling is the preservation of the epidermis, the protective layer of the skin. The publication entitled “Minimally Invasive Percutaneous Collagen Induction” by D. Fernandes, J Oral Maxillofac Surg Clin North Am. 2005 at 17:51-63, discloses the benefits of preserving the epidermis.

CN1137929A, CN202504281U and CN2166632 disclosed a cap with pocket filled with various herbs for promoting hair growth. A similar apparatus was disclosed in CN2091166U, wherein heating was believed helpful for increasing the absorption. CN106031534 disclosed a helmet with an internal lining with protrusions but it is not possible for the lining to conform to the scalp because the shape and size of heads are very different for different individuals. WO2006125367A1, EP3069762A1, CN202289237U, CN203609767U, CN203609768U, U.S. Pat. No. 6,666,878, U.S. Pat. Application 2012/00594440A1 disclosed caps with multiple laser diodes or light sources for hair growth. EP2486955A1 and CN203154620U disclosed cap with internal layer having affixed LED's to irradiate the scalp for promoting hair growth by stimulation of microcirculation and the analgesic effect or red light. EP0455422A2 disclosed a method of retarding or reversing baldness by applying growth factors or agents.

A plurality of inventions have previously recognized the desirability of stimulating the human scalp. A variety of different electro-mechanical devices have been proposed in the prior art (massage finger and vacuum, U.S. Pat. Nos. 4,469,092, 5,228,431, 6,228,041). It has been recognized that simple mechanical massaging is not satisfactory to retard the hair loss or promote hair growth. Apparatus using pulsating electrostatic fields (U.S. Pat. Nos. 5,595,564, 5,800,477, 6,834,206) and magnetic field (e.g. U.S. Pat. No. 6,267,720) have also been proposed. Hand held devices such as “lasercomb” (e.g. U.S. Pat. Nos. 8,771,327, 6,497,719, 7,201,764) have been disclosed also for stimulating hair growth. U.S. Pat. Nos. 7,819,828 and 4,744,350 relate to hand-held scalp roller massagers with different grip portion.

It is often desired to extend the length of stimulation or massage process in promoting hair growth, and it is inconvenient and tiring to use the hand-held devices. Therefore there is a need for a microneedling system can free the hand of the user. Furthermore, since the effect and mechanism is quite different for microneedling and light illumination, the improved system will work better to further include the effect of radiation.

### SUMMARY OF THE INVENTION

From the point of view of traditional or modern medicine, one of the ways for promoting hair growth is improving the blood flow in the scalp so that sufficient nutrition is supplied to the scalp and the functions could be activated or recovered. The present invention provides a microneedle headwear system or a cap with microneedles for mechanically perforating the outer skin layer and providing prolonged massage during the treatment. The presence of microneedles and massage action together allows for enhanced absorption of the active compounds, such as growth factors, when applied to the scalp. Even without the addition of active compound, a combined action of different stimulus or a broadened bandwidth of stimulation can reach maximized effect that any single action cannot achieve. The presence of illumination can further the effect of the cap or headwear for promoting hair growth. It is an object of the present invention to provide a microneedle system in the form of cap or headwear with at least one microneedle pad that can provide massage to the user's scalp that can improve the absorption of hair growth factors. It is another object of the present invention to further include the massage action by adding vibration means to facilitate the capillary blood flow and better absorption of growth factors and better transportation of nutrition to the scalp. It is yet another object of the present invention to apply the growth factor gradually to the scalp during the microneedling and massage action through capillary in order to prolong the absorption time. It is yet another object of the present invention to introduce light radiation during the microneedling and massage action to maximize the effect of promoting hair growth and harvest a combined results of microneedling, massage and light stimulation.

The present invention relates to a headwear or cap with at least one microneedle pad hanging inside of the cap. At least one vibration means powered by electrical source, such as a battery, is used to provide massage action to the scalp by the microneedle pad. When wear by the user, said microneedle pad conforms to the shape of users scalp and the microneedles reaches the scalp. Said microneedle pad is installed through an attaching means, such as a spring to the internal surface of the cap so said microneedle pad could vibrate freely to provide massage action through said vibration means. It is an object of the present invention to include a plurality of protrusions or fingers on the surface of said microneedle pad with at least one microneedle being on the tip of the finger of protrusion so the protrusions or fingers could reach the scalp through user's hair. It is another objective of the invention to provide the cap or headwear having at least one microneedle pad wherein said microneedle pad can be removed and replaced so the said microneedle pad can be cleaned, sanitized or replace when worn or when different length of microneedles is needed. It is yet another objective of the invention to provide the cap or headwear having at least one microneedle pad wherein said microneedle pad can be rearranged to fit the need of different hair loss pattern. It is yet another objective of the invention to provide the cap or headwear having at least two microneedle pads wherein said vibration means can be controlled so only predetermined microneedle pad will provide the massage action. It is yet another objective of the invention to provide the cap or headwear having at least one microneedle pad wherein said microneedle pad location can be rearranged to fit the need of different hair loss pattern. It is yet another objective of the invention to introduce light radiation powered by an electric source, such as a battery, to the scalp in the cap or headwear having microneedle pad. The radiation source can be selected from laser diode, light emitter diode (LED) or other light sources. The radiation source can be placed in between the microneedle pad and the internal surface of the cap. It is yet another objective of the invention to include a plurality of laser diodes or LED so large area of illumination to the user's scalp can be achieved during the microneedling and massage of the scalp. The radiation can reach the scalp through the microneedle pad or from the side of the microneedle pad.

### BRIEF DESCRIPTION OF THE DRAWINGS

FIG. 1 shows a hair loss treatment apparatus 101 in the form of a cap or hat.

FIG. 2 is an exploded view of an embodiment of the present invention.

FIG. 3 is another embodiment of the present invention, wherein the attaching means is flexible allowing the massage pad to cover a predetermined area of user's scalp during the massage and stimulation process.

FIG. 4 shows another embodiment of the present invention, wherein the vibration means is embedded in the cap and at least one microneedle massage pad is attached to the vibration means.

FIGS. 5A, 5B, and 5C show another preferred embodiment of the present invention, wherein each microneedle massage pad has its own vibration means and the position of said microneedle massage pad in the cap can be rearranged as required.

FIG. 6A-C show microneedle pad with protrusions that can go through the hair on user's scalp to reach the surface of scalp and to provide stimulation, and the location of said pad in the cap can be rearranged as needed.

FIG. 7 shows the views of another preferred embodiment wherein the microneedle massage pad having protrusions further includes a cavity holding growth factor, while the surface of protrusion contacting the user's scalp further includes at least one capillary hole connecting to said cavity.

FIG. 8A-B show the explosive views of an hair-loss treatment apparatus in FIG. 3, further includes a laser illumination means.

FIG. 9A-C show anther preferred embodiment wherein each microneedle massage pad in a hair loss treatment apparatus further includes its own laser diode illumination means.

### DETAILED DESCRIPTION OF THE INVENTION

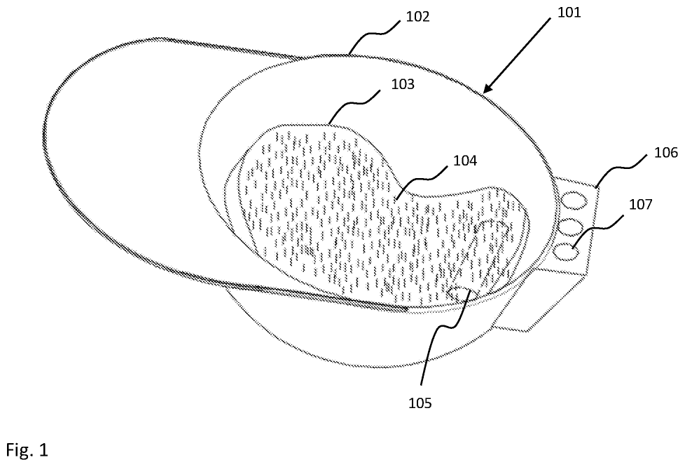

FIG. 1 shows a hair loss treatment apparatus 101 in the form of a cap or hat 102 having a massage pad 103 as the lining inside, said massage pad having a plurality of microneedles 104 of predetermined length on the surface contacting the user's scalp and at least one vibration means 105 powered by an electric source 106 such as a battery or external power source. Said massage pad is made of a soft or elastic material allowing itself to conform to the shape of the user's scalp while being firm enough to transmit the vibration action generated by said vibration means to the user's scalp, whereby said massage pad with microneedles can provide a massage action along with microneedling to stimulate the hair growth. A control means 107 can be used to control the level of vibration. The massage function can be prolonged while freeing the hands of the users. The external part of said cap or hat can be in any shape, form or thickness since it merely provides a cosmetic function and it also could help to hold said massage pad in place on the user's scalp.

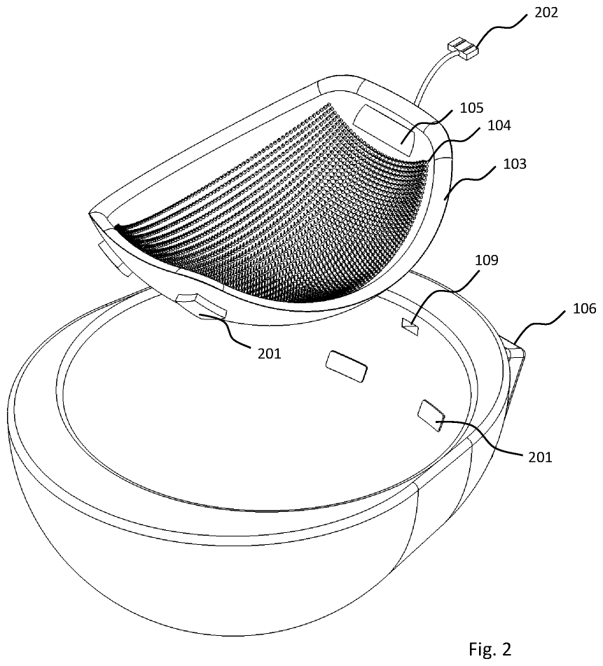

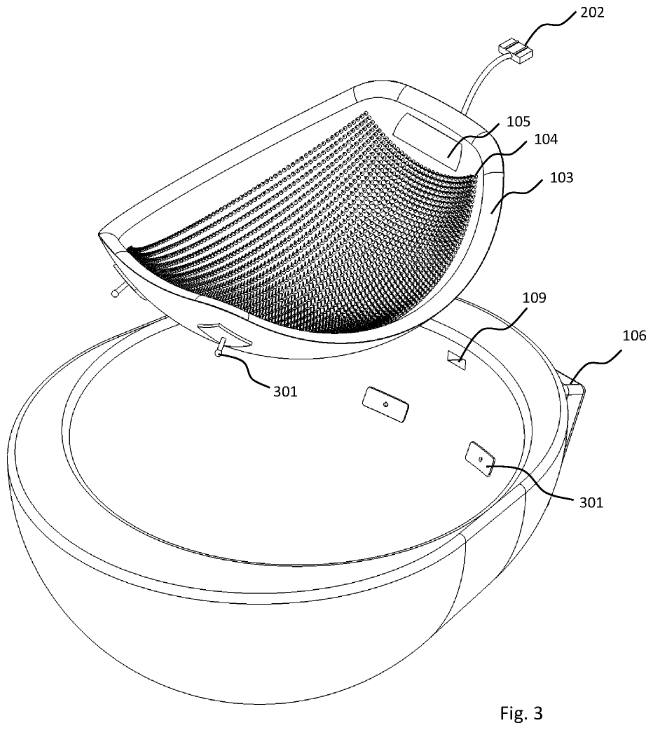

In one embodiment showing in FIG. 2, where said hair loss treatment apparatus is in a form of a cap for covering the user's scalp, having a massage pad 103 mounted on the internal lining of said cap, wherein said massage pad has a plurality of microneedles 104 of predetermined length on its surface contacting the user's scalp, in which said massage pad is removable. Said massage pad is attached to the inside of the cap with an attaching means 201. Said attaching means can be adhesive or a device such as a Velcro strip or a spring or a mechanical connection so said massage pad can be replaced, cleaned or changed to a different one with a different microneedle length. Said massage pad has at least one vibration means 105 inside providing massage action powered by a power source such as a battery 106 connected through connector 202 and 109. Said massage pad is made of a material such as silicon rubber or fabric cushion that is soft enough to conform to the shape of the user's scalp while firm or elastic enough to transmit the massage action caused by said vibration means to the user's scalp through said massage pad. In another embodiment in FIG. 3, the attaching means connecting said cap and said massage pad in FIG. 2 is at least one spring or a flexible string or elastic rod 301. Such flexible attaching means allows said massage pad to jump around on the user's scalp during vibration so each microneedle could cover a small area during the massage and stimulation process, whereby the treatment is carried out by combined action of vibrating massage pad and the stimulation of micro-needles to facilitate the blood flow of the user's scalp and increase the efficiency of drug delivery when medication is applied to the user's scalp.

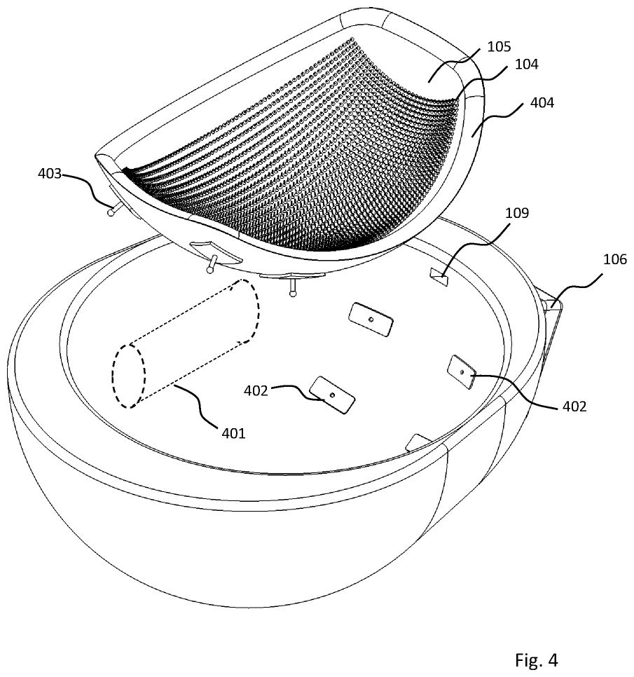

In another preferred embodiment showing in FIG. 4, where said hair loss treatment apparatus is similar to the device in FIG. 3 with at least one microneedle massage pad 404, but in this embodiment, the vibration means 401 powered by a power source such as a battery 106 is embedded in the cap instead of inside the microneedle massage pad. Through internal connections said vibration means sends vibration force to a plurality of attaching locations 402 where said microneedle massage pad 404 can be attached using the attaching means 403. In this embodiment, said microneedle massage pad being removable has a plurality of microneedles 104 of predetermined length on its surface contacting the user's scalp. Said attaching means can be adhesive or a device such as a Velcro strip or a spring or a mechanical connection that can be attach to selected attaching location 402 to transmit the vibration action of said vibration means to said microneedle massage pad and then to the scalp of user's scalp. Thus said microneedle massage pad can be replaced, cleaned or changed to a different one with a different microneedle length. Said massage pad is made of a material such as silicon rubber or fabric cushion that is soft enough to conform to the shape of the user's scalp while firm or elastic enough to transmit the massage action caused by said vibration means to the user's scalp through said massage pad.

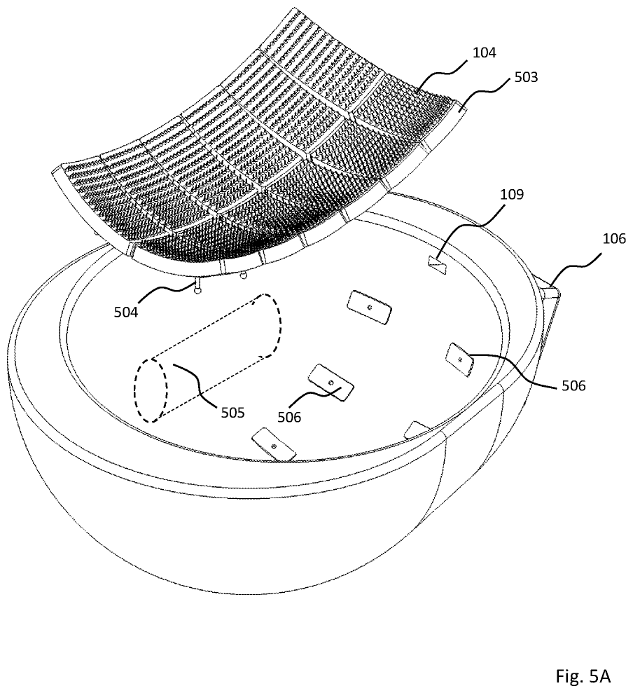

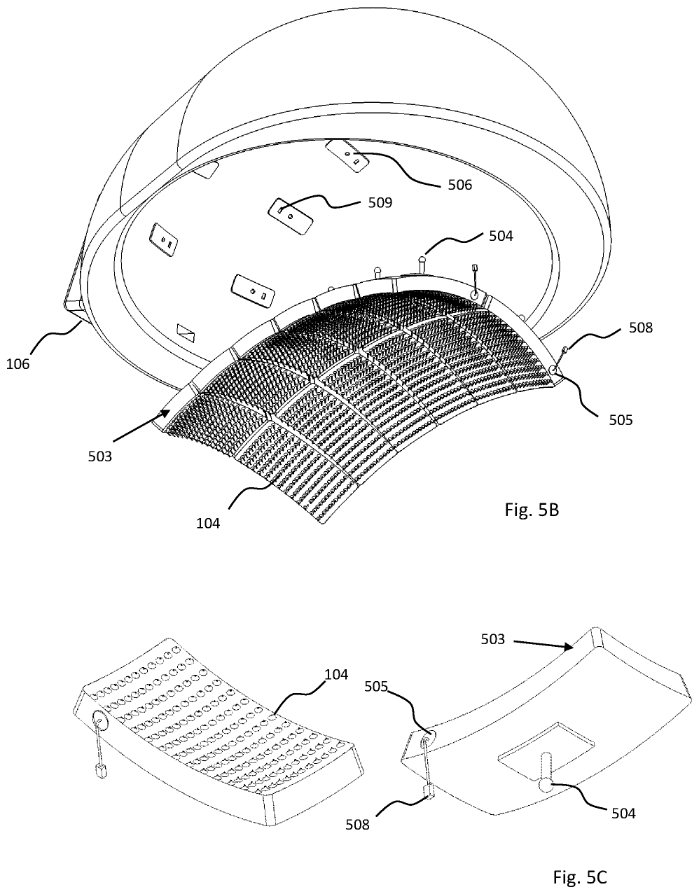

In a further embodiment of the invention in FIG. 5, where said hair loss treatment apparatus is in a form of a cap for covering the user's scalp, having multiple massage pads 503 and each said massage pad attached to the internal lining of said cap with a attaching means 504, wherein each said massage pad has a plurality of microneedles 104 of predetermined length on its surface contacting the user's scalp. The massage action is provided by a vibration means 505 embedded in said cap powered by an electric source 106. Said vibration means having a plurality of connection means (not shown) can to send vibration action through said connection means to each massage pad through attaching locations 506 as in FIG. 5A, or each massage pad has its own embedded vibration means 505 and power connection 508 as in FIG. 5B and FIG. 5C. Power connection sockets 509 are distributed inside of said cap for power connection of 508. In this embodiment said massage pads 503 are removable from said cap and the location of each said pad can be rearranged as need. When a large area of scalp is covered, multiple massage pads could conform to the shape of the user's head better. Said attaching means can be adhesive or a device such as a Velcro strip or a spring or a mechanical connection so said massage pad can be replaced, cleaned or changed to a different one having a different microneedle length. Said massage pad is made of a material such as silicon rubber or fabric cushion that is soft enough to conform to the shape of the user's scalp while firm or elastic enough to transmit the massage action caused by said vibration means to the user's scalp through said massage pad.

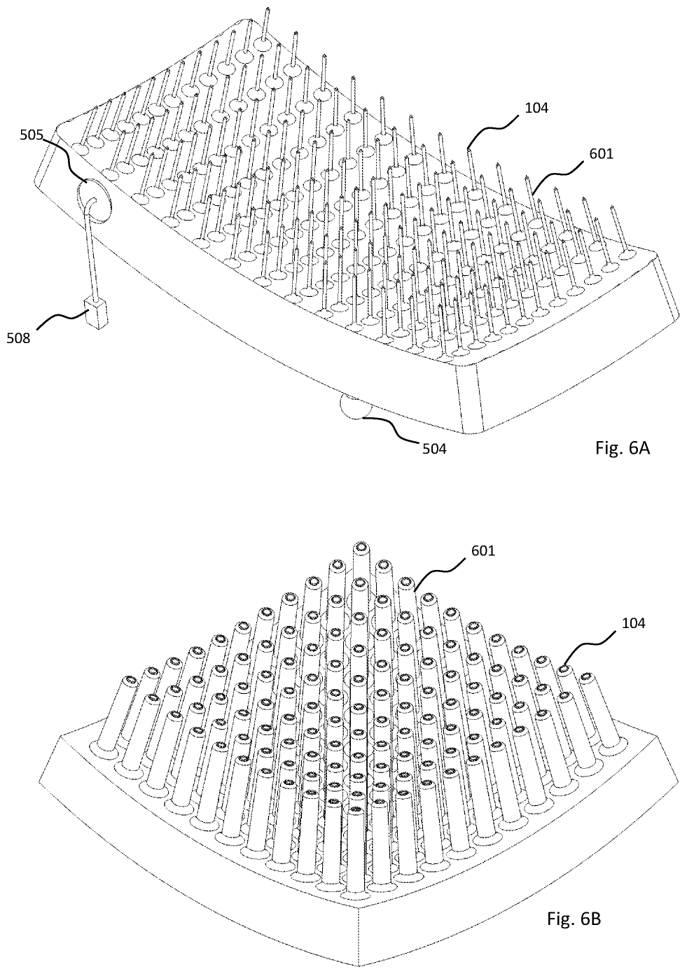

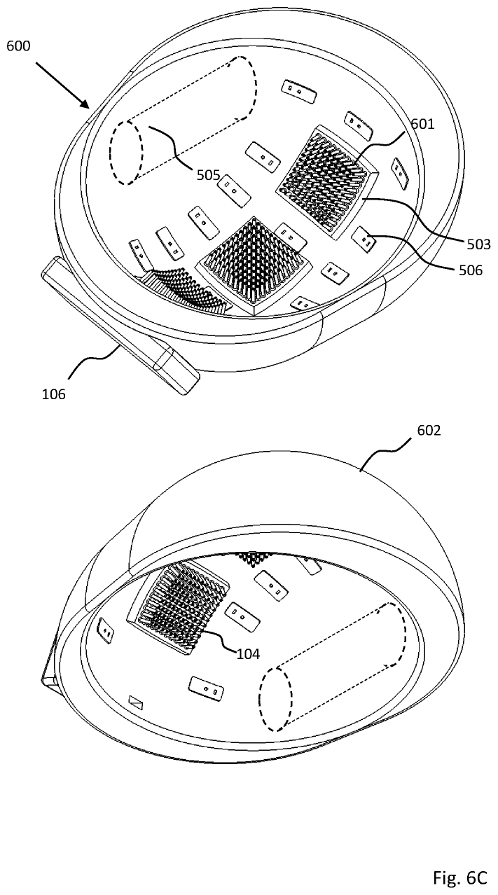

FIGS. 6A and 6B shows embodiments wherein the massage pad has a plurality of protrusions 601 and each protrusion has at least one microneedle on the tip. Preferably it has several microneedles on the tip. The protrusions, like the teeth of a comb, could easily go through the hair of the user, and make the microneedles easier to reach the user's scalp. This feature will help users with partial hair loss. FIG. 6C shows two views of a hair loss treatment apparatus 600 comprising a cap 602 with multiple microneedle massage pads 503 that can be rearranged according to the need, and each massage pad can be detached from the cap for replacement and cleaning.

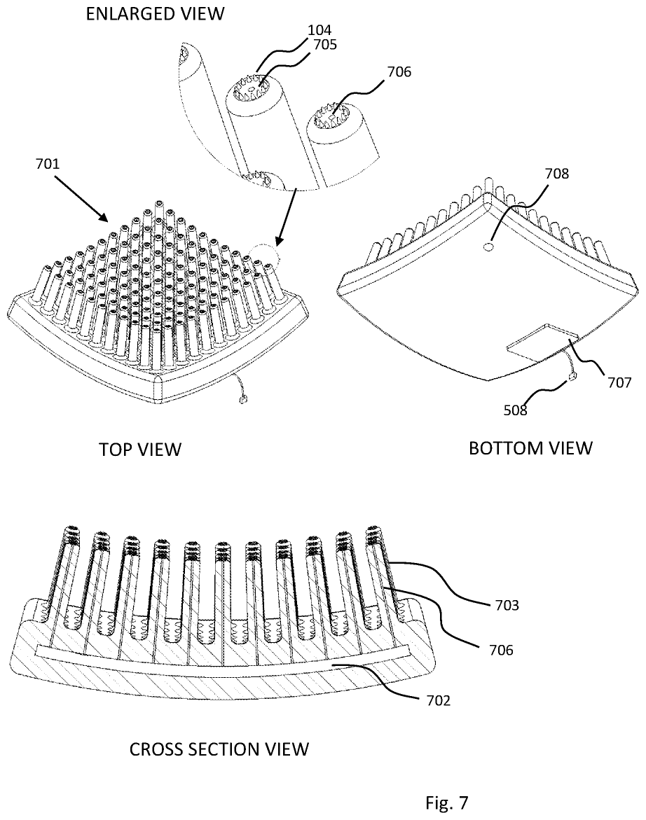

Hair growth factors or agents can be used together with the microneedle cap disclosed in this invention. These could be applied to the user's scalp before using the hair-loss treatment apparatus. One problem in using hair growth factors or agents has been that the applied layer dries in short time and it is inconvenient to re-apply, particularly when there is hair on the scalp. FIG. 7 shows a solution provided by the current invention wherein the massage pad 701, having protrusions 703 with microneedles 104 similar to FIG. 6B, further includes a cavity 702 holding growth factor, and the surface 705 contacting the user's scalp of said massage pad 701, such as said the tips of protrusions 703, further includes at least one capillary hole 706 connecting to said cavity 702, whereby said growth factor is gradually and slowly applied to the user's scalp during the massage action by the vibration means 707, and can be refilled from port 708. The speed of application can be controlled by the hole size of the capillary.

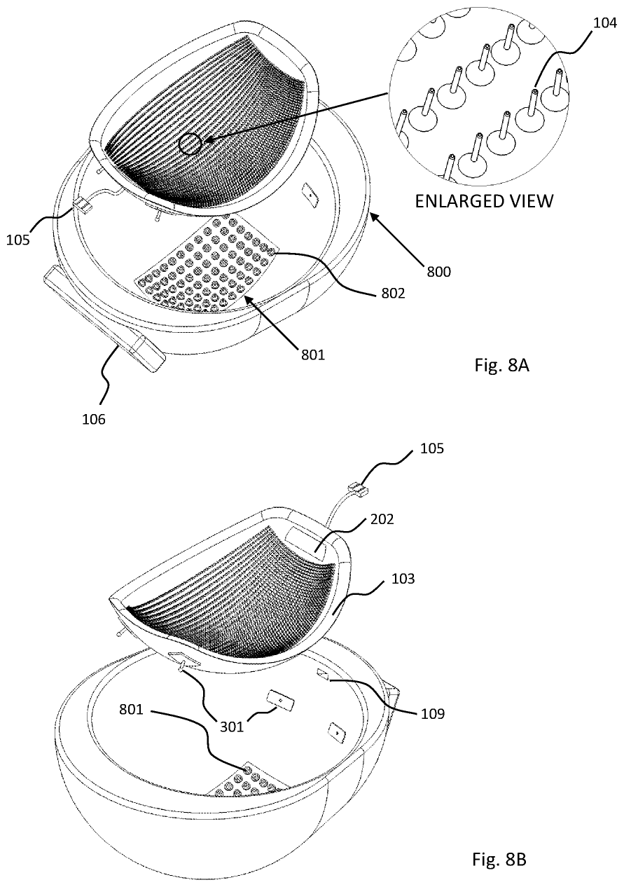

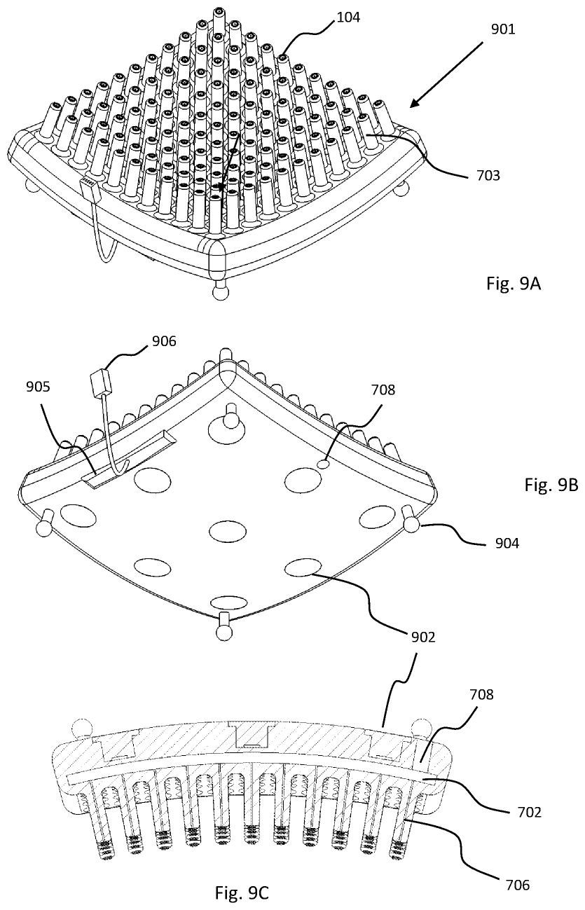

To further promoting the hair growth, assisting options can be added to the microneedle cap. One preferred embodiment is shown in FIG. 8, wherein the said hair-loss treatment apparatus in FIG. 3 further includes a laser illumination means 801 placed in between said massage pad 103 and the inside lining of the cap. The laser illumination means 801 consisting a plurality of laser diodes 802 provides a broad area laser illumination to the scalp through said massage pad 103 and/or through the gap between the microneedle pads. The microneedle pad is made of transparent or translucent materials so the laser power will reach the scalp efficiently directly or by scattering. It is preferred to use a laser with visible or near-infrared wavelength for illumination. It is more preferably to select one or more wavelength band from a range between 500-1900 nm. It is even more preferably to select at least one band between 630-680 nm. Many materials are transparent to the wavelength can be used for making the microneedle pad. The microneedles 104 on the surface contacting the user's scalp are very tiny so illumination will not be affected. In yet another embodiment showing in FIG. 9, a laser illumination means comprising at least one laser diode powered by an electrical source is mounted on the microneedle massage pad, providing local area laser illumination to the scalp. By using a pad like 901, the microneedle massage pad location can be rearranged to work on different areas of the user's scalp as need as the embodiment in FIG. 6. This can be easily achieved by plugging the microneedle massage pad 901 through the power connector 906 for getting power for illumination means 902 and vibration means 905 from the power source such as a battery (not shown). 901 can be mounted to a cap such as 206 through the attaching means 904, which is similar as 504. 901 is similar to 701 having a cavity for hair growth factor, if needed. The laser illumination, the massage action and the microneedling along with the application of hair growth factors of the disclosed hand-free cap will maximize the purpose of promoting hair growth.

The foregoing descriptions of embodiments of the invention have been presented for the purpose of illustration and description. It is not intended to limit the invention to the precise form disclosed, and obviously many modification and variation are possible in light of above teaching. For example, optics can be used in certain cases to change the divergence of the illumination means for covering different size of user's scalp. Light source other than laser diodes can be used also to provide needed wavelength for illumination. Pressure means such as a cap holder can be used to make pressurized contact of the massage pad with the user's scalp. The cap can be modified as headwear like a helmet, a hat, or other shape. Moreover, this microneedling procedure is universal. When made flexible, as it can easily be, it is possible to use a large pad to cover other part of the body for stimulation and treatment. A string or other restrain means can be added to hold the cap or hat in place and add pressure of the microneedle massage pad to the user's scalp. The embodiments were chosen and described in order to best explain the principles of the invention and its practical applications to thereby enable others skilled in the art to best utilize the present invention. While specific values have been used and listed in the foregoing embodiments for the sake of easy to understand, they do not set limit on the invention teaching, which is properly described in the claims. The particular values and configurations discussed above can be varied and are cited merely to illustrate particular embodiments of the present invention and are not intended to limit the scope of the present invention.

What is claimed is:

1. A hair-loss treatment apparatus, comprising
a cap covering the user's scalp,
at least one massage pad attached inside said cap by an attaching means, wherein said massage pad has a plurality of micro-needles of predetermined length on its surface contacting the user's scalp,
a vibration means attached to said massage pad powered by an electrical source providing massage action to the user's scalp,
whereby the treatment is carried out by combined action of vibrating massage pad and the stimulation of micro-needles to facilitate the blood flow of the user's scalp and increase the efficiency of drug delivery when medication is applied to the user's scalp.

2. A hair-loss treatment apparatus as in claim 1, said massage pad having a plurality of protrusions wherein each protrusion has at least one micro-needle on the tip reaching the scalp through existing hairs.

3. A hair-loss treatment apparatus as in claim 1, said massage pad can be detached from the cap for replacement and cleaning.

4. A hair-loss treatment apparatus as in claim 2, wherein said massage pad further includes a cavity holding growth factor, and said protrusion further include a capillary hole on the tip connecting to said cavity, whereby said growth factor is applied to the user's scalp during the massage action.

5. A hair-loss treatment apparatus as in claim 1, further including a laser illumination means placed in between said massage pad and the inside of the cap, wherein laser illumination means providing a broad area laser illumination to the scalp through said massage pad.

6. A hair-loss treatment apparatus as in claim 1, further including a laser illumination means mounted on said massage pad, providing local area laser illumination to the scalp.

7. A hair-loss treatment apparatus as in claim 5, wherein said massage pad is made of material transparent to the wavelength of the laser illumination.

8. A hair-loss treatment apparatus as in claim 5, wherein the wavelength of the laser illumination is at least one band selected between 500-1900 nm.

9. A hair-loss treatment apparatus as in claim 5, wherein the wavelength of the laser illumination is at least one band selected between 630-680 nm.

10. A hair-loss treatment apparatus as in claim 5, wherein the laser illumination means comprising at least one laser diode powered by said electrical source.

11. A hair-loss treatment apparatus, comprising
a cap covering the user's scalp having at least one electrical connector providing electrical power and attaching means on the internal surface of said cap.
at least one massage pad attached inside said cap through said attaching means, wherein said massage pad has a plurality of micro-needles of predetermined length on its surface contacting the user's scalp,
a vibration means attached to said massage pad drawing power through said electrical connectors and providing massage action to the user's scalp, wherein said massage pad can be plugged inside said cap in a predetermined pattern to suit different hair loss user, whereby the treatment is carried out by combined action of vibrating massage pad and the stimulation of micro-needles to facilitate the blood flow of the user's scalp and increase the efficiency of drug delivery when medication is applied to the user's scalp.

12. A hair-loss treatment apparatus as in claim 11, said massage pad having a plurality of protrusions wherein each protrusion has at least one micro-needle on the tip reaching the scalp through existing hairs.

13. A hair-loss treatment apparatus as in claim 11, said massage pad can be detached from the cap for replacement and cleaning.

14. A hair-loss treatment apparatus as in claim 12, wherein said massage pad further includes a cavity holding growth factor, and said protrusion further include a capillary hole on the tip connecting to said cavity, whereby said growth factor is applied to the user's scalp during the massage action.

15. A hair-loss treatment apparatus as in claim 11, further including a laser illumination means placed in between said massage pad and the inside of the cap, wherein laser illumination means providing a broad area laser illumination to the scalp through said massage pad.

16. A hair-loss treatment apparatus as in claim 11, wherein each massage pad further including a laser illumination means, providing local area laser illumination to the scalp.

17. A hair-loss treatment apparatus as in claim 15, wherein said massage pad is made of material transparent to the wavelength of the laser illumination.

18. A hair-loss treatment apparatus as in claim 15, wherein the wavelength of the laser illumination is at least one band selected between 500-1900 nm.

19. A hair-loss treatment apparatus as in claim 15, wherein the wavelength of the laser illumination is at least one band selected between 630-680 nm.

20. A hair-loss treatment apparatus, comprising
a cap covering the user's scalp having at least one embedded vibration means powered by a power source,
at least one attaching means on the internal surface of said cap, said attaching means interconnecting to said vibration means,
at least one massage pad attached inside of said cap by said attaching means, wherein said massage pad has a plurality of micro-needles of predetermined length on its surface contacting the user's scalp,
whereby the treatment is carried out by combined action of vibrating massage pad and the stimulation of micro-needles to facilitate the blood flow of the user's scalp and increase the efficiency of drug delivery when medication is applied to the user's scalp.
